(19) United States
(12) Patent Application Publication
(10) Pub. No.: US 2013/0115246 A1
(43) Pub. Date: May 9, 2013

(54) REDUCED DOSE ORAL PHARMACEUTICAL COMPOSITIONS OF FENOFIBRATE

(71) Applicants: Venkat Reddy KALLEM, Pune (IN); Raghu Rami Reddy KASU, Pune (IN); Subhasis DAS, Pune (IN); Vijaya Kumar THOMMANDRU, Pune (IN); Ninad DESHPANDAY, Pune (IN)

(72) Inventors: Venkat Reddy KALLEM, Pune (IN); Raghu Rami Reddy KASU, Pune (IN); Subhasis DAS, Pune (IN); Vijaya Kumar THOMMANDRU, Pune (IN); Ninad DESHPANDAY, Pune (IN)

(73) Assignee: Lupin Atlantis Holdings, S.A., Schaffhausen (CH)

(21) Appl. No.: 13/350,099

(22) Filed: Jan. 13, 2012

(30) Foreign Application Priority Data
Nov. 5, 2011 (IN) 1426/KOL/2011

(51) Int. Cl. A61K 9/14; A61P 3/00; A61K 31/216; A61P 3/06; B82Y 5/00; B82B 1/00

(52) U.S. Cl. 424/400; 514/543; 977/773; 977/915

(57) ABSTRACT

The invention relates to reduced dose oral pharmaceutical composition of fenofibrate which exhibits substantial bioequivalence to Antara® Capsules under fasting condition and also capable of reducing the food effect on bioavailability of fenofibrate. Provided is a pharmaceutical composition comprising about 90 mg of fenofibrate particles having a D₉₀ particle size of less than about 600 nm and a pharmaceutically acceptable carrier, wherein the pharmaceutical composition is a solid dosage form suitable for oral administration and is substantially free of food effect such that when administered orally to a human provides an AUC₀₋ₜ value for fenofibric acid in the blood plasma of the human under a fed state which is higher than the AUC₀₋ₜ value under a fasted state by up to 12%, wherein t is 96 hours from the administration of the pharmaceutical composition.

This application claims benefit of Serial No. 1426/KOL/2011, filed 5 Nov. 2011 in India and which application is incorporated herein by reference in its entirety.

### FIELD OF THE INVENTION

The invention relates to reduced dose oral pharmaceutical composition of fenofibrate which exhibits substantial bioequivalence to Antara® Capsules under fasting condition and also capable of reducing the food effect on bioavailability of fenofibrate. The invention provides a method of treatment of hyperlipidemias, hypercholesterolemias and/or hypertriglyceridemias in a patient by administering reduced dose pharmaceutical composition of fenofibrate with or without food and a process of manufacturing the composition.

### BACKGROUND OF THE INVENTION

Fibrates are lipid regulating agents. Examples of fibrates include fenofibrate, bezafibrate, clofibrate and ciprofibrate. The compounds are regarded as prodrugs and are metabolised in vivo to their active metabolites. For illustrative purposes only, the following is based on a specific example of a fibrate, namely fenofibrate. Fenofibrate is chemically named as 2-[4-(4-chlorobenzoyl)phenoxy]-2-methyl-propanoic acid, 1-methylethyl ester. Fenofibrate is metabolised to the active substance fenofibric acid. Fenofibric acid has an elimination half-life of about 20 hours.

Fenofibric acid is the active metabolite of fenofibrate which leads to reduction in total cholesterol, LDL cholesterol, apolipoprotein B, total triglycerides, and triglyceride rich lipoprotein (VLDL) in treated patients. In addition, treatment with fenofibrate results in increased high density lipoprotein (HDL) and apoproteins apo AI and apo AII. Fenofibrate acts as a potent lipid regulating agent offering unique and clinical advantages over existing products in the fibrate family of drug substances. Fenofibrate produces substantial reduction in plasma triglyceride levels in hypertriglyceridemic patients and in plasma cholesterol and LDL-C in hypercholesterolemic and mixed dyslipidemic patients.

Clinical studies have demonstrated that elevated levels of total cholesterol, low density lipoprotein cholesterol (LDL-C), and apo-lipoprotein B (apo B) are associated with human atherosclerosis. Decreased levels of high density lipoprotein cholesterol (HDL-C) and its transport complex, apolipoprotein A (apo AI and apo AII) are associated with the development of atherosclerosis. Fenofibrate is also effective in the treatment of Diabetes Type II and metabolic syndrome. Fenofibrate is also indicated as adjunctive therapy to diet for treatment of adult patients with hypertriglyceridemia (Fredrickson Types IV and V hyperlipedemia).

Fibrates are drug substances known to be poorly and variably absorbed after oral administration. Normally fibrates are prescribed to be taken with food in order to increase the bioavailability. Fenofibrate is very poorly soluble in water, and the absorption of which in the digestive tract is limited. An increase in its solubility or in its rate of solubilization leads to better digestive absorption. Therefore a number of improvements have been made in an effort to improve the bioavailability and efficacy of currently approved fenofibrate dosage forms. Various approaches such as micronization of the fenofibrate, addition of a surfactant, and co-micronization of fenofibrate with a surfactant have been explored in order to increase the rate of solubilization of fenofibrate.

WO 2010/082214 discloses a fenofibrate formulation with enhanced oral bioavailability comprising fenofibrate dissolved in a lipophilic surfactant. It also discloses that such formulation at lower doses may improve side effect profile.

US 2007/0014846 discloses compositions, particularly, pharmaceutical compositions in particulate form such as granulate or in solid dosage forms comprising a combination of a fibrate and a statin. More specifically, it discloses a solid pharmaceutical composition comprising atorvastatin and a low dose, i.e. a reduced amount, of fenofibrate having improved bioavailability and/or improved pharmacological response, i.e. improved effect.

US 2004/0057999 discloses an orally administrable fenofibrate tablet, wherein the required daily dose is lower than 200 mg.

However, there still exists a need for pharmaceutical composition which is capable of reducing the food effect on the bioavailability of fenofibrate. Furthermore, there is also a need for reduced dose oral pharmaceutical composition of fenofibrate that reduces side-effects.

The present invention relates to reduced dose oral pharmaceutical composition of fenofibrate which exhibits substantial bioequivalence to Antara® Capsules under fasting condition and also capable of reducing the food effect on bioavailability of fenofibrate.

### SUMMARY OF THE INVENTION

The present invention relates to a reduced dose oral pharmaceutical composition of fenofibrate.

An embodiment of the invention encompasses a nanoparticulate pharmaceutical composition comprising about 90 mg of fenofibrate and a pharmaceutically acceptable carrier. The nanoparticulate fenofibrate particles have particle size of less than about 3000 nm.

Yet another embodiment of the invention directs a nanoparticulate pharmaceutical composition comprising about 90 mg of fenofibrate and a pharmaceutically acceptable carrier wherein the pharmacokinetic profile of the fenofibrate is substantially free of food effect when administered orally to a human, wherein the pharmacokinetic profile is defined by Cₘₐₓ and AUC.

Yet another embodiment discloses a nanoparticulate pharmaceutical composition comprising about 90 mg of fenofibrate and a pharmaceutically acceptable carrier, wherein composition exhibits substantial bioequivalence to Antara® Capsules under fasting condition.

Another embodiment of the invention is directed to a nanoparticulate pharmaceutical composition comprising about 90 mg of fenofibrate and a pharmaceutically acceptable carrier, wherein the composition exhibits improved pharmacokinetic profile as compared to Antara® Capsules under fed condition, wherein the pharmacokinetic profile is defined by Cₘₐₓ and AUC.

Yet another embodiment discloses a nanoparticulate pharmaceutical composition comprising about 90 mg of fenofibrate and a pharmaceutically acceptable carrier, wherein the composition exhibits a mean AUC_{(96 hrs)} of about 114073.70 ng.h/ml under fasting condition and a mean AUC_{(96 hrs)} of about 123327.66 ng.h/ml under fed condition.

Yet another embodiment discloses a nanoparticulate pharmaceutical composition comprising about 90 mg of fenofibrate and a pharmaceutically acceptable carrier, wherein the composition exhibits a mean Cₘₐₓ of about 7326.84 ng/ml under fasting condition and a mean Cₘₐₓ of about 6866.43 ng/ml under fed condition.

Yet another embodiment discloses a method of treating a patient in need of treatment for primary hyperlipidemias, hypercholesterolemias and/or hypertriglyceridemias comprising administering to the patient reduced dose oral pharmaceutical composition of fenofibrate.

### BRIEF DESCRIPTION OF DRAWING

FIG. 1: Represents the comparative plasma level of fenofibric acid of Antara® 130 mg capsule under fasting and fenofibrate composition of Example 2 under fasting and fed condition.

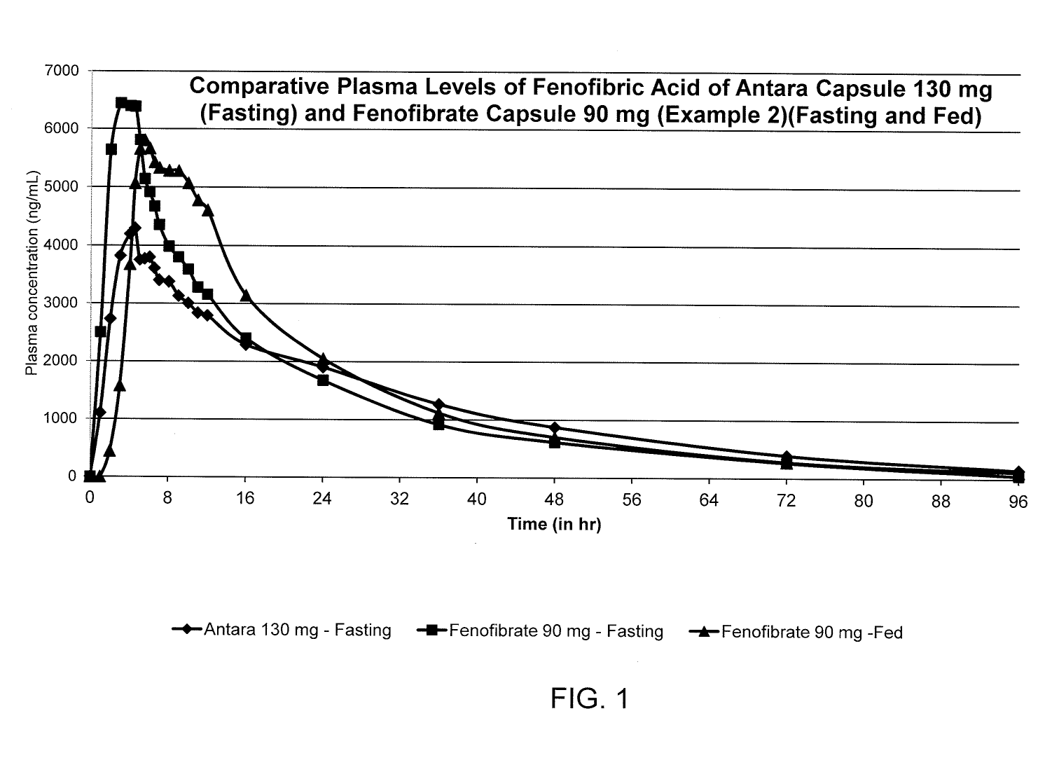

### DETAILED DESCRIPTION OF THE INVENTION

The specification discloses reduced dose oral pharmaceutical composition of fenofibrate which is capable of reducing the food effect on the bioavailability of fenofibrate. The composition exhibits substantial bioequivalence to Antara® Capsules under fasting condition. The composition makes it effective at lower doses as well as improves high dose associated side effect profile of fenofibrate. The composition also offers a method of treatment of primary hyperlipidemias, hypercholesterolemias and/or hypertriglyceridemias comprising administering reduced dose oral pharmaceutical composition of fenofibrate to the patient with or without food. The specification discloses a process of manufacturing reduced dose oral pharmaceutical compositions of fenofibrate.

“Fenofibrate” as employed herein refers to fenofibrate, its derivatives, prodrugs, active metabolites, and/or its polymorphs, solvates, hydrates, enantiomers, racemates and mixtures thereof. Further, it also includes amorphous or crystalline polymorphic forms of fenofibrate, and mixtures thereof. Fenofibrate for the purpose of the invention is used in micronized form or in nanoparticulate form or combination thereof. The nanoparticulate fenofibrate, particles may have particle size of less than about 3000 nm. The term “particle size of less than about 3000 nm”, is meant that the 90% of the fenofibrate particles have a particle size less than about 600 nm and preferably less than about 500 nm.

The USFDA has approved fenofibrate tablet as well as capsule which contain different dose of fenofibrate. Lipidil® Capsule of Abbott contained 100 mg of fenofibrate. Tricor Micronised® Capsule of Abbott contained 67, 134 and 200 mg of fenofibrate. Tricor® Tablet of Abbott contained 54 and 160 mg of fenofibrate. The above mentioned dosage forms have been discontinued by Abbott. The prescription dosage form of fenofibrate such as Tricor® Tablet of Abbott contains 48 and 145 mg, Lipophen® Capsule of Cipher contains 50, 100 and 150 mg, Triglide ® Tablet of Skyepharma contains 50 and 160 mg, Fenoglide® Tablet of Sciele Pharma contains 40 and 120 mg, Antara® Capsule of Lupin Atlantis contains 43 and 130 mg of fenofibrate.

In general, it is known that the absorption and bioavailability of drug substance can be affected by a variety of factors when administered orally. Such factors include the presence of food in the gastrointestinal tract and, in general, the gastric residence time of a drug substance is significantly longer in the presence of food than in the fasted state. If the bioavailability of a drug substance is affected beyond a certain point due to the presence of food in the gastrointestinal tract, the drug substance is said to exhibit a food effect. Food effects are important because there is a risk associated with administering the drug substance to a patient who has eaten recently. The risk derives from the potential that absorption into the bloodstream may be adversely affected to the point that the patient risks insufficient absorption to remedy the condition for which the drug was administered. The pharmacokinetic studies of Antara® Capsule disclose that the extent of absorption of fenofibric acid was unaffected when Antara® was taken either in fasted state or with a low-fat meal. However, the Cmax of Antara® is increased in the presence of a low-fat meal. Tₘₐₓ was unaffected in the presence of a low-fat meal. In the presence of a high-fat meal, there was a 26% increase in AUC and 108% increase in C. of fenofibric acid from Antara® relative to fasting state.

Accordingly, an embodiment of the present invention provides a pharmaceutical composition comprising about 90 mg of fenofibrate particles having a D₉₀ particle size of less than about 600 nm and a pharmaceutically acceptable carrier, wherein the pharmaceutical composition is a solid dosage form suitable for oral administration and is substantially free of food effect such that when administered orally to a human provides an AUC₀₋ₜ value for fenofibric acid in the blood plasma of the human under a fed state which is higher than the AUC₀₋ₜ value under a fasted state by up to 12%, wherein t is 96 hours from the administration of the pharmaceutical composition, preferably an AUC₀₋ₜ value for fenofibric acid in the blood plasma of the human under a fed state which is higher than the AUC₀₋ₜ value under a fasted state by up to 10% and more preferably an AUC₀₋ₜ value for fenofibric acid in the blood plasma of the human under a fed state which is higher than the AUC₀₋ₜ value under a fasted state by about 7% to about 9%. However it is further preferred that the D₅₀ particle size of fenofibrate is less than about 200 nm.

Another embodiment of the invention, a pharmaceutical composition comprising about 90 mg of fenofibrate particles having a D₉₀ particle size of less than about 600 nm and a pharmaceutically acceptable carrier, wherein the pharmaceutical composition provides a Cₘₐₓ value for fenofibric acid in the blood plasma of the human under a fasted state which is higher than the Cₘₐₓ value under a fed state by less than 9%, preferably less than 8% and most preferably by about 5% to about 7%. However it is further preferred that the D₅₀ particle size of fenofibrate is less than about 200 nm.

Accordingly, a method of reducing food effect is provided when treating hyperlipidemias, hypercholesterolemias and hypertriglyceridemias in a patient, comprising administering to the patient reduced dose oral pharmaceutical composition of fenofibrate. The specification discloses oral pharmaceutical composition of 30 or 90 mg of fenofibrate which exhibits substantial bioequivalence to Antara® 43 and 130 mg Capsule when dosed under fasted conditions.

Yet another embodiment provides AUC₀₋ₜ of the pharmaceutical composition of the present invention value under the fasted state, and AUC₀₋ₜ value of Antara® 130 mg Capsule under the fasted state does not vary by 10%, preferably by 7%, more preferably by 5% and most preferably by 2%.

The term “reduced dose” used herein refers to the low dose relative to Antara® 43 and 130 mg Capsule that is 30 and 90 mg of fenofibrate respectively.

The term “bioavailability” denotes the degree to which a drug substance becomes available to the target tissue after administration.

As used herein, the term “bioequivalence” denotes a scientific basis on which two or more pharmaceutical compositions containing same active ingredient are compared with one another. “Bioequivalence” means the absence of a significant difference in the rate and extent to which the active agent in pharmaceutical equivalents or pharmaceutical alternatives becomes available at the site of action when administered in an appropriately designed study. Bioequivalence can be determined by an in vivo study comparing a pharmacokinetic parameter for the two compositions. Parameters often used in bioequivalence studies are Tmax, Cmax, AUC_{0-inf}, AUC₀₋ₜ. In the present context, substantial bioequivalence of two compositions is established by 90% confidence intervals (CI) of between 0.80 and 1.25 for AUC.

In a specific embodiment, substantial bioequivalence of the reduced dose oral pharmaceutical composition of fenofibrate with Antara® Capsule under fasting condition is determined according to the Federal Drug Administration's (FDA) and the corresponding European regulatory agency (EMEA) guidelines and criteria.

The term “Tₘₐₓ” denotes the time to reach the maximal plasma concentration (Cₘₐₓ) after administration; AUC_{0-inf} or AUC denotes the area under the plasma concentration versus time curve from time 0 to infinity; AUC₀₋ₜ denotes the area under the plasma concentration versus time curve from time 0 to time t. For statistical analysis of pharmacokinetic data, the logarithmic transformed AUC₀₋ₜ, AUC_{0-∞}, or Cₘₐₓ data can be analyzed statistically using analysis of variance.

The terms “without food” and “fasted” are equivalent and are as given by FDA guidelines and criteria. The term “fasted” means the condition wherein no food is consumed within 1 hour prior to administration of the composition or 2 hours after administration of the composition.

Summary of Relative Bioavailability Studies A comparison of the relative bioavailability of 90 mg fenofibrate composition prepared according to example 2 and Antara® 130 mg Capsules was carried out in 12 healthy volunteers under fasted conditions.

In these examples, “fasted” is based on a 10-hour absence of food; however, a skilled artisan would know other methods of preparing fasted conditions. For example, “fasted” may be understood as 10 hour or more absence of food.

Conditions for fasted state were according to Guidance for Industry: Food-effect Bioavailability and Fed Bioequivalence Studies; CDER December 2002: An overnight fast of the subjects of at least 10 hours; no breakfast and no food intake 4 hours after drug administration; 240 ml plain water at study drug administration.

Results of relative bioavailability studies under fasting and fed conditions are as indicated in the Tables below:

TABLE 1
Represents the results of relative bioavailability
studies under fasting conditions
Comparative Bioavailability Results Under Fasting Condition (N = 12)

| Fenofibrate 90 mg Capsule (Example 2) | | | Antara ® 130 mg Capsule | | |
|---|---|---|---|---|---|
| ^{#}AUC₀₋ₜ | ^{#}Cₘₐₓ | *Tₘₐₓ | ^{#}AUC₀₋ₜ | ^{#}Cₘₐₓ | *Tₘₐₓ |
| (ng · h/ml) | (ng/ml) | (hours) | (ng · h/ml) | (ng/ml) | (hours) |
| 114073.70 | 7326.84 | 3.00 | 113006.43 | 4480.10 | 4.50 |

Statistical Parameters

| Parameters | Cₘₐₓ | AUC₀₋ₜ |
|---|---|---|
| Least Square Mean Ratio (%) | 163.54 | 100.94 |
| 90% Confidence Interval | 145.93-183.26 | 95.50-106.69 |
| Intra-subject CV (%) | 16.24 | 7.86 |

^{#}Least square means
*Median

The results demonstrate that the oral pharmaceutical composition containing 90 mg fenofibrate exhibits substantial bioequivalence (based on AUC) to Antara® 130 mg Capsule under fasting conditions.

In the same study the effect of food on the bioavailability of oral pharmaceutical compositions containing 90 mg fenofibrate, prepared according to example 2 was evaluated in 11 healthy volunteers.

TABLE 2
Represents the result of food effect on the bioavailability of oral
pharmaceutical composition containing 90 mg fenofibrate (Example 2)
Food effect on Fenofibrate capsule 90 mg (N = 11)

| Fenofibrate 90 mg Capsule | | | Fenofibrate 90 mg Capsule | | |
|---|---|---|---|---|---|
| (Under High Fat Fed Condition) | | | (Under Fasting Condition) | | |
| ^{#}AUC₀₋ₜ | ^{#}Cₘₐₓ | *Tₘₐₓ | ^{#}AUC₀₋ₜ | ^{#}Cₘₐₓ | *Tₘₐₓ |
| (ng · h/ml) | (ng/ml) | (hours) | (ng · h/ml) | (ng/ml) | (hours) |
| 123327.66 | 6866.43 | 5.50 | 114073.70 | 7326.84 | 3.00 |

Statistical Parameters

| Parameters | Cₘₐₓ | AUC₀₋ₜ |
|---|---|---|
| Least Square Mean Ratio (%) | 93.72 | 108.11 |
| 90% Confidence Interval | 83.35-105.38 | 102.09-114.50 |
| Intra-subject CV (%) | 16.24 | 7.86 |

^{#}Least square means
*Median

Results of Table 1 demonstrate that oral pharmaceutical composition containing 90 mg fenofibrate exhibits substantial bioequivalence with Antara Capsules 130 mg under fasting condition.

Results of Table 2 indicate no food effect on the bioavailability of oral pharmaceutical composition containing 90 mg fenofibrate.

The oral pharmaceutical composition containing 90 mg fenofibrate shows about 63% increase in Cₘₐₓ compared to Antara Capsules 130 mg under fasting condition.

The oral pharmaceutical composition containing 90 mg fenofibrate shows about 6% decrease in Cmax and 8% increase in AUC for fenofibric acid in the presence of high fat meal. However, Antara Capsules reportedly showed 26% and 108% increase in AUC and Cmax, respectively, under high fat fed condition.

The reduced dose oral pharmaceutical composition includes, but not limited to granules, grains, beads or pellets, minitablets which are filled into capsules or sachets or are compressed to tablets by conventional methods. The granules, grains, beads or pellets are optionally enteric-coated or coated with a protective coating.

The term “pharmaceutically acceptable carrier” is intended to denote any material, which is inert in the sense that it substantially does not have any therapeutic and/or prophylactic effect. Such carrier may be added with the purpose of making it possible to obtain a pharmaceutical composition, which has acceptable technical properties. The pharmaceutical composition of the invention may contain one or more pharmaceutically acceptable carrier.

Examples of suitable carrier for use in a composition according to the invention include fillers, diluents, binders, disintegrants, stabilizers, lubricants, antifoaming agents or mixtures thereof.

Fillers or diluents, which include, but are not limited to compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used. Filler or diluents can also function as inert carrier. The individual particle size of the inert carrier can be between 50 and 500 micron.

Binders include, but not limited to hydrophilic polymer. The term “hydrophilic polymer” used herein mean any high molecular weight substance (greater, for example, than 300) having sufficient affinity towards water to dissolve therein and form a gel. Examples of such polymers are polyvinylpyrrolidone, poly(vinyl alcohol), hydroxypropylcellulose, hydroxymethylcellulose, hydroxypropylmethylcellulose (HPMC), gelatin, etc. Polymer blends are also suitable. The preferred hydrophilic polymer is HPMC. The HPMC used in this invention has, for example, a molecular weight comprised between 5000 and 60,000, preferably for example between 10,000 and 30,000.

Specific examples of disintegrants includes, but are not limited to alginic acid or alginates, microcrystalline cellulose, hydroxypropyl cellulose and other cellulose derivatives, croscarmellose sodium (Ac-di-sol), crospovidone, polacrillin potassium, sodium starch glycolate, starch, pregelatinized starch, carboxymethyl starch (e.g. Primogel® and Explotab®).

Glidants and lubricants include, but are not limited to stearic acid, magnesium stearate, calcium stearate or other metallic stearate, talc, waxes and glycerides, light mineral oil, polyethylene glycols, glyceryl behenate, colloidal silica, hydrogenated vegetable oils, sodium stearyl fumarate.

Stabilizers include but are not limited to, surface-active agents. Any surfactant is suitable, whether it be amphoteric, non-ionic, cationic or anionic. Examples of such surfactants are: sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, polyoxyethylene fatty acid glycerides, Poloxamer®, etc. Mixtures of surfactants are also suitable. The preferred surfactant is sodium laurylsulfate, which can be co-micronized with fenofibrate.

Antifoaming agents include, but are not limited to simethicone emulsion, dimethicone emulsion.

The reduce dose oral pharmaceutical composition may be prepared by any known technique in the art but not limited to wet granulation, melt granulation and dry granulation. The preferred method is wet granulation which includes low shear wet granulation; high shear wet granulation and fluid bed granulation. The most preferred method according to the invention uses the fluidized bed granulation principle. In particular, the invention employs the micronized fenofibrate alone or fenofibrate comicronized with surfactant. The comicronized Fenofibrate-surfactant mixture can be subjected to various processes such as wet milling, high-pressure homogenization, emulsification, precipitation, rapid expansion, and spray freezing to produce fenofibrate nanoparticles. The preferred method is wet milling using suitable mill such as DYNO mill.

The suspension of the fenofibrate in a nanoparticulate form in a solution of a hydrophilic polymer and, optionally, a surfactant and antifoaming agent, is sprayed onto the inert cores. For the purpose of making fenofibrate suspension, solvents like aqueous or organic (for example ethanol) can be used. Purified water is preferred.

The granules thus obtained can, if desired, be provided with a coating which can be lubricated and filled into hard gelatin capsule or can be compressed into tablets.

When the granules obtained (whether subsequently coated or not) is compressed to form tablets, this step can be implemented using any conventional technique which is suitable, for example using an alternating or rotating compressing equipment.

It must be noted that as used in the specification and the appended claims, the singular forms also include the plural unless the context clearly dictates otherwise.

The examples below are representation only and should not be construed to limit the scope of the invention:

### EXAMPLE 1

| Sr. No. | Ingredients | Qty (mg/cap) |
|---|---|---|
| 1 | Fenofibrate (Micronized) | 90.00 |
| 2 | Sodium lauryl sulphate | 5.40 |
| 3 | Hydroxypropyl Methylcellulose | 20.769 |
| | (3 cps) | |
| 4 | Simethicone Emulsion | 0.415 |
| 5 | Purified water | qs |
| 6 | Sugar Spheres | 17.03 |
| 7 | Talc | 1.386 |

| | |
|---|---|
| Total weight | 134.585 |

Brief Manufacturing Procedure

Step I. Nanonization of Fenofibrate:
1. Steadily add Hydroxypropyl Methylcellulose to purified water while stirring until to form a clear solution.
2. Add sodium lauryl sulphate (SLS) to the step-1 under constant stirring.
3. Add fenofibrate (Micronized) to the step-2 under constant stirring.
4. Add simethicone emulsion to the suspension of step 3 and stir slowly to form a uniform suspension.
5. Filter the suspension of step 4 through mesh #100 ASTM.
6. Pass the suspension of step 5 through DYNO MILL till the desired particle size is obtained.

Step II. Drug Loading:
7. Load the sugar spheres in the fluidized bed processor and pre warm to the product bed temperature of 40±5° C.
8. Spray the suspension of step 6 on the sugar spheres of step 7.
9. Sift the fenofibrate loaded pellets through mesh #14 ASTM.

Step III. Lubrication:
10. Sift talc through mesh #40 ASTM and mix with the pellets of step 9 by using suitable blender for 5 minutes for lubrication.

Step IV. Capsule Filling:
11. Fill the final pellets of step 10 into a suitable capsule shell.

### EXAMPLE 2

| Sr. No. | Ingredients | Qty (mg/cap) |
|---|---|---|
| 1 | Fenofibrate (Micronized) | 90.00 |
| 2 | Sodium lauryl sulphate | 5.40 |
| 3 | Hydroxypropyl Methylcellulose | 23.00 |
| | (3 cps) | |
| 4 | Simethicone Emulsion | 0.415 |
| 5 | Purified water | qs |
| 6 | Sugar Spheres | 14.799 |
| 7 | Talc | 1.386 |

| | |
|---|---|
| Total weight | 135.00 |

Brief Manufacturing Procedure

Step I. Nanonization of Fenofibrate:
1. Steadily add Hydroxypropyl Methylcellulose to purified water while stirring until to form a clear solution.
2. Add sodium lauryl sulphate (SLS) to the step-1 under constant stirring.
3. Add fenofibrate (Micronized) to the step-2 under constant stirring.
4. Add simethicone emulsion to the suspension of step 3 and stir slowly to form a uniform suspension.
5. Filter the suspension of step 4 through mesh #100 ASTM.
6. Pass the suspension of step 5 through DYNO MILL till the desired particle size is obtained.

Step II. Drug Loading:
7. Load the sugar spheres in the fluidized bed processor and pre warm to the product bed temperature of 40±5° C.
8. Spray the suspension of step 6 on the sugar spheres of step 7.
9. Sift the fenofibrate loaded pellets through mesh #14 ASTM.

Step III. Lubrication:
10. Sift talc through mesh #40 ASTM and mix with the pellets of step 9 by using suitable blender for 5 minutes for lubrication.

Step IV. Capsule Filling:
11. Fill the final pellets of step 10 into a suitable capsule shell. Alternatively, fill the final pellets of step 10 for 30 mg composition of fenofibrate in a capsule of suitable size.

### EXAMPLE 3

| Sr. No. | Ingredients | Qty (mg/cap) |
|---|---|---|
| 1 | Fenofibrate (Micronized) | 90.00 |
| 2 | Sodium lauryl sulphate | 5.40 |
| 3 | Hydroxypropyl Methylcellulose | 20.77 |
| | (3 cps) | |
| 4 | Simethicone Emulsion | 0.42 |
| 5 | Purified water | qs |
| 6 | Sugar Spheres | 62.03 |
| 7 | Talc | 1.389 |

| | |
|---|---|
| Total weight | 180.00 |

Brief Manufacturing Procedure

Step I. Nanonization of Fenofibrate:
1. Steadily add Hydroxypropyl Methylcellulose to purified water while stirring until to form a clear solution.
2. Add sodium lauryl sulphate (SLS) to the step-1 under constant stirring.
3. Add fenofibrate (Micronized) to the step-2 under constant stirring.
4. Add simethicone emulsion to the suspension of step 3 and stir slowly to form a uniform suspension.
5. Filter the suspension of step 4 through mesh #100 ASTM.
6. Pass the suspension of step 5 through DYNO MILL till the desired particle size is obtained.

Step II. Drug Loading:
7. Load the sugar spheres in the fluidized bed processor and pre warm to the product bed temperature of 40±5° C.
8. Spray the suspension of step 6 on the sugar spheres of step 7.
9. Sift the fenofibrate loaded pellets through mesh #14 ASTM.

Step III. Lubrication:
10. Sift talc through mesh # 40 ASTM and mix with the pellets of step 9 by using suitable blender for 5 minutes for lubrication.

Step IV. Capsule Filling:
11. Fill the final pellets of step 10 into a suitable capsule shell.

### EXAMPLE 4

| Sr. No. | Ingredients | Qty (mg/cap) |
|---|---|---|
| 1 | Fenofibrate (Micronized) | 90.00 |
| 2 | Sodium lauryl sulphate | 5.40 |
| 3 | Hydroxypropyl Methylcellulose (3cps) | 22.00 |
| 4 | Simethicone Emulsion | 0.42 |
| 5 | Purified water | qs |
| 6 | Sugar Spheres | 13.100 |
| 7 | Hydroxypropyl Methylcellulose (E5) | 2.16 |
| 8 | Talc | 1.93 |

| | |
|---|---|
| Total weight | 135.0 |

Brief Manufacturing Procedure

Step I. Nanonization of Fenofibrate:
1. Steadily add Hydroxypropyl Methylcellulose to purified water while stirring until to form a clear solution.
2. Add sodium lauryl sulphate (SLS) to the step-1 under constant stirring.
3. Add fenofibrate (Micronized) to the step-2 under constant stirring.
4. Add simethicone emulsion to the suspension of step 3 and stir slowly to form a uniform suspension.
5. Filter the suspension of step 4 through mesh #100 ASTM.
6. Pass the suspension of step 5 through DYNO MILL till the desired particle size is obtained.

Step II. Drug Loading:
7. Load the sugar spheres in the fluidized bed processor and pre warm to the product bed temperature of 40±5° C.
8. Spray the suspension of step 6 on the sugar spheres of step 7.
9. Sift the fenofibrate loaded pellets through mesh #14 ASTM.

Step III. Coating:
10. Add Hydroxypropyl Methylcellulose (HPMC E5) to purified water while stirring until to form a clear solution.
11. Add required quantity of talc to step-10 under constant stirring slowly to form a uniform dispersion.
12. Filter the dispersion of step 11 through mesh #100 ASTM.
13. Coat the step-12 dispersion on step-9 pellets in the fluidized bed processor. Step III. Lubrication:
14. Sift talc through mesh #40 ASTM and mix with the pellets of step 13 by using suitable blender for 5 minutes for lubrication.

Step IV. Capsule Filling:
15. Fill the final pellets of step 14 into a suitable capsule shell.

### EXAMPLE 5

| Sr. No. | Ingredients | Qty (mg/cap) |
|---|---|---|
| 1 | Fenofibrate (Micronized) | 90.00 |
| 2 | Sodium lauryl sulphate | 2.7 |
| 3 | Hydroxypropyl Methylcellulose (3cps) | 20.00 |
| 4 | Simethicone Emulsion | 0.42 |
| 5 | Purified water | qs |
| 6 | Sugar Spheres | 58.30 |
| 7 | Hydroxypropyl Methylcellulose (E5) | 5.76 |
| 8 | Talc | 2.83 |

| | |
|---|---|
| Total weight | 180.0 |

Brief Manufacturing Procedure

Step I. Nanonization of Fenofibrate:
1. Steadily add Hydroxypropyl Methylcellulose to purified water while stirring until to form a clear solution.
2. Add sodium lauryl sulphate (SLS) to the step-1 under constant stirring.
3. Add fenofibrate (Micronized) to the step-2 under constant stirring.
4. Add simethicone emulsion to the suspension of step 3 and stir slowly to form a uniform suspension.
5. Filter the suspension of step 4 through mesh #100 ASTM.
6. Pass the suspension of step 5 through DYNO MILL till the desired particle size is obtained.

Step II. Drug Loading:
7. Load the sugar spheres in the fluidized bed processor and pre warm to the product bed temperature of 40±5° C.
8. Spray the suspension of step 6 on the sugar spheres of step 7.
9. Sift the fenofibrate loaded pellets through mesh #14 ASTM.

Step III. Coating:
10. Add Hydroxypropyl Methylcellulose (HPMC E5) to purified water while stirring until to form a clear solution.
11. Add required quantity of talc to step-10 under constant stirring slowly to form a uniform dispersion.
12. Filter the dispersion of step 11 through mesh #100 ASTM.
13. Coat the step-12 dispersion on step-9 pellets in the fluidized bed processor.

Step III. Lubrication:
14. Sift talc through mesh #40 ASTM and mix with the pellets of step 13 by using suitable blender for 5 minutes for lubrication.

Step IV. Capsule Filling:
15. Fill the final pellets of step 14 into a suitable capsule shell.

### EXAMPLE 6

| Sr. No. | Ingredients | Qty (mg/cap) |
|---|---|---|
| 1 | Fenofibrate-SLS (Co-micronized) | 95.36 |
| 2 | Hydroxypropyl Methylcellulose | 20.77 |
| | (3 cps) | |
| 3 | Simethicone Emulsion | 0.42 |
| 4 | Purified water | qs |
| 5 | Sugar Spheres | 17.07 |
| 6 | Talc | 1.39 |

| | |
|---|---|
| Total weight | 135.0 |

Brief Manufacturing Procedure

Step I. Nanonization of Fenofibrate:
1. Steadily add Hydroxypropyl Methylcellulose to purified water while stirring until to form a clear solution.
2. Add fenofibrate-sodium lauryl sulphate (SLS) mixture to the step-1 under constant stirring.
3. Add simethicone emulsion to the suspension of step 2 and stir slowly to form a uniform suspension.
4. Filter the suspension of step 3 through mesh #100 ASTM.
5. Pass the suspension of step 4 through DYNO MILL till the desired particle size is obtained.

Step II. Drug Loading:
6. Load the sugar spheres in the fluidized bed processor and pre warm to the product bed temperature of 40±5° C.
7. Spray the suspension of step 5 on the sugar spheres of step 6.
8. Sift the fenofibrate loaded pellets through mesh #14 ASTM.

Step III. Lubrication:
9. Sift talc through mesh #40 ASTM and mix with the pellets of step 8 by using suitable blender for 5 minutes for lubrication.

Step IV. Capsule Filling:
10. Fill the final pellets of step 9 into a suitable capsule shell.

### EXAMPLE 7

| Sr. No. | Ingredients | Qty (mg/cap) |
|---|---|---|
| 1 | Fenofibrate-SLS (Co-micronized) | 95.36 |
| 2 | Hydroxypropyl Methylcellulose | 26.35 |
| | (3 cps) | |
| 3 | Simethicone Emulsion | 0.42 |
| 4 | Purified water | qs |
| 5 | Sugar Spheres | 31.49 |
| 6 | Talc | 1.39 |

| | |
|---|---|
| Total weight | 155 |

Brief Manufacturing Procedure

Step I. Nanonization of Fenofibrate:
1. Steadily add Hydroxypropyl Methylcellulose to purified water while stirring until to form a clear solution.
2. Add fenofibrate-sodium lauryl sulphate (SLS) mixture to the step-1 under constant stirring.
3. Add simethicone emulsion to the suspension of step 2 and stir slowly to form a uniform suspension.
4. Filter the suspension of step 3 through mesh #100 ASTM.
5. Pass the suspension of step 4 through DYNO MILL till the desired particle size is obtained.

Step II. Drug Loading:
6. Load the sugar spheres in the fluidized bed processor and pre warm to the product bed temperature of 40±5° C.
7. Spray the suspension of step 5 on the sugar spheres of step 6.
8. Sift the fenofibrate loaded pellets through mesh #14 ASTM.

Step III. Lubrication:
9. Sift talc through mesh #40 ASTM and add mix with the pellets of step 8 by using suitable blender for 5 minutes for lubrication.

Step IV. Capsule Filling:
10. Fill the final pellets of step 9 into a suitable capsule shell.

1. A pharmaceutical composition comprising about 90 mg of fenofibrate particles having a D₉₀ particle size of less than about 600 nm and a pharmaceutically acceptable carrier, wherein the pharmaceutical composition is a solid dosage form suitable for oral administration and is substantially free of food effect such that when administered orally to a human provides an AUC₀₋ₜ value for fenofibric acid in the blood plasma of the human under a fed state which is higher than the AUC₀₋ₜ value under a fasted state by up to 12%, wherein t is 96 hours from the administration of the pharmaceutical composition.

2. The pharmaceutical composition of claim 1, wherein the D₉₀ particle size of the fenofibrate particles is about 500 nm.

3. The pharmaceutical composition of claim 1, wherein the D₅₀ particle size of the fenofibrate particles is about 100 to about 200 nm.

4. The pharmaceutical composition of claim 1, wherein the D₅₀ particle size of the fenofibrate particles is about 120 to about 180 nm.

5. The pharmaceutical composition of claim 1, wherein the AUC₀₋ₜ under the fed state is higher than the AUC₀₋ₜ value under the fasted state by up to 10%.

6. The pharmaceutical composition of claim 1, wherein the AUC₀₋ₜ under the fed state is higher than the AUC₀₋ₜ value under the fasted state by about 7% to about 9%.

7. The pharmaceutical composition of claim 1, which provides a Cₘₐₓ value for fenofibric acid in the blood plasma of the human under a fasted state which is higher than the Cₘₐₓ value under a fed state by less than 9%.

8. The pharmaceutical composition of claim 1, which provides a Cₘₐₓ value for fenofibric acid in the blood plasma of the human under a fasted state which is higher than the Cₘₐₓ value under a fed state by less than 8%.

9. The pharmaceutical composition of claim 1, which provides a Cₘₐₓ value for fenofibric acid in the blood plasma of the human under a fasted state which is higher than the Cₘₐₓ value under a fed state by about 5% to about 7%.

10. The pharmaceutical composition of claim 1, which is substantially bioequivalent under the fasted state to a second pharmaceutical composition in solid dosage form and suitable for oral administration comprising 130 mg of micronized fenofibrate particles having a D₉₀ of less than 15 μm but greater than 600 nm and D₅₀ of less than 7 μm but greater than 200 nm and a pharmaceutical carrier such that the AUC₀₋ₜ value under the fasted state of the pharmaceutical composition is substantially the same as the AUC₀₋ₜ value under the fasted state of the second pharmaceutical composition.

11. The pharmaceutical composition of claim 10, wherein the AUC₀₋ₜ value of the pharmaceutical composition under the fasted state and of the second pharmaceutical composition does not vary by 10%.

12. The pharmaceutical composition of claim 10, the AUC₀₋ₜ value of the pharmaceutical composition under the fasted state and of the second pharmaceutical composition does not vary by 7%.

13. The pharmaceutical composition of claim 10, wherein the AUC₀₋ₜ value of the pharmaceutical composition under the fasted state and of the second pharmaceutical composition does not vary by 5%.

14. The pharmaceutical composition of claim 10, wherein the AUC₀₋ₜ value of the pharmaceutical composition under the fasted state and of the second pharmaceutical composition does not vary by 2%.

15. The pharmaceutical composition of claim 1, wherein the solid dosage form is a capsule.

16. A method of treating a patient for primary hyperlipidemia, hypercholesterolemia, and/or hypertriglyceridemia comprising administering to the patient the pharmaceutical composition of claim 1.
